(19) United States
(12) Patent Application Publication
(10) Pub. No.: US 2025/0340760 A1
(43) Pub. Date: Nov. 6, 2025

(54) ADHESIVE COMPOSITIONS CONTAINING MAGNETIC PARTICLES

(71) Applicant: UNIVERSITY OF KANSAS, Lawrence, KS (US)

(72) Inventors: Cory BERKLAND, Wildwood, MO (US); Jian QIAN, Lawrence, MO (US)

(21) Appl. No.: 18/880,062

(22) PCT Filed: Jun. 29, 2023
(86) PCT No.: PCT/US2023/026559

§ 371 Date: Dec. 30, 2024

Related U.S. Application Data
(60) Provisional application No. 63/356,769, filed on Jun. 29, 2022.

(51) Int. Cl. C09J 11/04; A61K 49/18; A61L 26/00; C08K 9/06; C09J 5/00; C09J 135/04; C09J 151/02; C09J 163/00; C09J 181/02; H01F 1/20

(52) U.S. Cl. CPC C09J 11/04; A61K 49/1818; A61L 26/0014; A61L 26/0019; A61L 26/0061; C09J 5/00; C09J 135/04; C09J 151/02; C09J 163/00; C09J 181/02; H01F 1/20; C08K 9/06

(57) ABSTRACT

Described herein are magnetic adhesive compositions. The adhesive compositions can comprise a curable matrix comprising one or more reactive precursor molecules, and a population of magnetic particles dispersed within the curable matrix. The magnetic particles can each comprise a magnetic particle core and shell at least partially encapsulating the magnetic particle core. The magnetic particles can be present in an effective amount to induce and direct flow of the adhesive composition under an applied magnetic field. As a consequence, a magnetic field can be used to control application and/or curing of these adhesive composition at a desired location. For example, these adhesive compositions can be applied as a flowable fluid, and subsequently directed to flow to a desired location prior to curing (and/or held at a desired location during curing). Accordingly, also provided are methods of adhering surfaces using these adhesive compositions.

### CROSS-REFERENCE TO RELATED APPLICATION

This application claims priority to U.S. Provisional Application 63/356,769, filed on Jun. 29, 2022, the contents of which is hereby incorporated in its entirety.

### BACKGROUND

Cyanoacrylates are a family of strong fast-acting adhesives with industrial, medical, and household applications. The acryl groups rapidly polymerize in the presence of water to form long, strong chains. Specific cyanoacrylates include methyl 2-cyanoacrylate (MCA), ethyl 2-cyanoacrylate (ECA, commonly sold under trade names such as “Super Glue” and “Krazy Glue”, or Toagosei), n-butyl cyanoacrylate (n-BCA), octyl cyanoacrylate and 2-octyl cyanoacrylate (used in medical, veterinary and first aid applications).

Superglues, such as various forms of cyanoacrylates, often do not flow into hard to reach locations. Conversely, properly placed superglues can leak out of the desired location for adhesion. Typically, the addition of magnetic particles produce agglomerated or precipitated particles or cure the superglues. Superglues come in a variety of formulations and viscosities. For example, cyanoacrylates can exhibit viscosities near that of water while octyl-cyanoacylates are much more viscous. Also, polymers are often added as viscosity modifiers to achieve flow properties desired for the particular application.

In medical applications several different techniques have been used for internal wound closure and tissue fixation such as sutures, tapes, staples, ligating clips, and adhesives. Cyanoacrylate adhesives are liquid monomers that can polymerize creating a mechanical barrier, which maintains a natural healing environment. Because their polymerization is initiated by functional groups found on biomolecules in tissue, their tissue bonding capability is greater than other types of adhesives. However, the delivery of cyanoacrylates into hard to reach areas is limited due to their rapid cure time. Once placed, cyanoacrylates can leak out of target sites.

Therefore, there is a need for adhesives that can easily flow into hard to reach locations and be held in place prior to polymerization.

### SUMMARY

Described herein are magnetic adhesive compositions. The adhesive compositions can comprise a curable matrix comprising one or more reactive precursor molecules, and a population of magnetic particles dispersed within the curable matrix. The magnetic particles can each comprise a magnetic particle core and shell at least partially encapsulating the magnetic particle core.

The magnetic particles can be present in an effective amount to induce and direct flow of the adhesive composition under an applied magnetic field. As a consequence, a magnetic field can be used to control application and/or curing of these adhesive compositions at a desired location. For example, these adhesive compositions can be applied as a flowable fluid, and subsequently directed to flow to a desired location prior to curing (and/or held at a desired location during curing). In some examples, the magnetic field can be applied to direct the flowable fluid to a hard-to-reach location prior to curing (e.g., a location to which the adhesive composition cannot be directly applied, for example, due to spatial constraints). In some embodiments, the magnetic field can be applied to retain the adhesive at a desired location during curing of the adhesive composition (e.g., to prevent an adhesive from leaking, dislodging, or washing away prior to curing).

Accordingly, also provided are methods of applying the adhesive compositions described herein. For example, disclosed are methods for adhering a first surface and a second surface at a location that comprise applying an adhesive composition described herein as a flowable magnetic fluid to a locus in proximity to the first surface, the second surface or a combination thereof; applying a magnetic field for a period of time effective to direct the flowable magnetic fluid to the location and/or to immobilize the flowable magnetic fluid at the location; and curing the adhesive composition. Also disclosed are methods of sealing a wound comprising applying an adhesive composition described herein as a flowable magnetic fluid to a locus in proximity to a tissue surface in proximity to the wound; applying a magnetic field for a period of time effective to direct the flowable magnetic fluid towards the wound and/or to immobilize the flowable magnetic fluid in place at the wound; and curing the adhesive composition to seal the wound. Optionally, these methods can further involve imaging to confirm placement of the cured adhesive composition.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a diagram of a surface alkyl medication of silica coated magnetic nanoparticles. The diagram of a suspendable/dispersible and does not cure glue magnetic particle structure is shown. The silica coating protects from curing the glues. The octyl-mnp modifies the surface so the magnetic particles suspend in the glues.

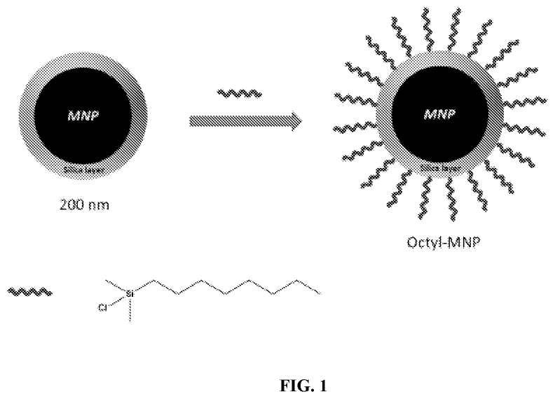

FIG. 2 are images of octyl-MNP in 3M vetbond when the magnetic field applied (2A) and removed (2B). While the magnetic field was applied the magnetic glue was held by magnetic field offsetting the effect of gravity. The magnetic glue flowed under the effect of gravity without the magnetic field.

FIG. 3 are images of octyl-MNP in Chenso when the magnetic field applied (3A) and removed (3B). While the magnetic field was applied the magnetic glue was held by magnetic field offsetting the effect of gravity. The magnetic glue flowed under the effect of gravity without the magnetic field.

FIG. 4A-4B are images of octyl-MNP in Dermabond when the magnetic field applied (4A) and removed (4B). While the magnetic field was applied the magnetic glue was held by magnetic field offsetting the effect of gravity. The magnetic glue flowed under the effect of gravity without the magnetic field.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

### General Definitions

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “hardening” or “curing” a composition are used interchangeably and refer to polymerization and/or crosslinking reactions including, for example, photopolymerization reactions and chemical polymerization techniques (e. g., ionic reactions or chemical reactions forming radicals effective to polymerize ethylenically unsaturated compounds) involving one or more materials included in the composition.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

“Effective concentration”, as used herein, generally refer to a concentration of the surface modified magnetic particle evenly dispersed in the flowable magnetic fluid sufficient to allow a magnetic field to hold the flowable magnetic fluid in place during curing.

“Shell”, as used herein, generally refer to, a coating, a layer, a barrier, an encapsulating material that can partially or completely encapsulates (surrounds) the magnetic particle core. The shell can be inert (and by extension render the magnetic particles inert), meaning that the magnetic particles do not induce curing of the curable matrix in the absence of light, air, and moisture.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —S¹Z¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

### Adhesive Compositions

Described herein are magnetic adhesive compositions that comprise a curable matrix and a population of magnetic particles dispersed within the curable matrix. The curable matrix can comprise one or more reactive precursor molecules, as discussed in more detail below.

The magnetic particles can be present in an effective amount to induce and direct flow of the adhesive composition under an applied magnetic field. As a consequence, a magnetic field can be used to control application and/or curing of these adhesive compositions at a desired location. For example, these adhesive compositions can be applied as a flowable fluid, and subsequently directed to flow to a desired location prior to curing (and/or held at a desired location during curing). In some examples, the magnetic field can be applied to direct the flowable fluid to a hard-to-reach location prior to curing (e.g., a location to which the adhesive composition cannot be directly applied, for example, due to spatial constraints). In some embodiments, the magnetic field can be applied to retain the adhesive at a desired location during curing of the adhesive composition (e.g., to prevent an adhesive from being dislodged or washed away prior to curing).

In some embodiments, the magnetic particles can be present in the adhesive composition in an amount of at least 0.1% by weight (e.g., at least 0.5% by weight, at least 1% by weight, at least 2% by weight, at least 2.5% by weight, at least 3% by weight, at least 4% by weight, at least 5% by weight, at least 6% by weight, at least 7% by weight, at least 7.5% by weight, at least 8% by weight, at least 9% by weight, at least 10% by weight, at least 15% by weight, at least 20% by weight, or at least 25% by weight), based on the total weight of the adhesive composition. In some embodiments, the magnetic particles can be present in the adhesive composition in an amount of 30% by weight or less (e.g., 25% by weight or less, 20% by weight or less, 15% by weight or less, 10% by weight or less, 9% by weight or less, 8% by weight or less, 7.5% by weight or less, 7% by weight or less, 6% by weight or less, 5% by weight or less, 4% by weight or less, 3% by weight or less, 2.5% by weight or less, 2% by weight or less, 1% by weight or less, or 0.5% by weight or less), based on the total weight of the adhesive composition.

The quantity of magnetic particles present in the adhesive composition can range from any of the minimum values described above to any of the maximum values described above. For example, in some embodiments, the magnetic particles can be present in the adhesive composition in an amount of from 0.1% by weight to 30% by weight, such as from 2.5% by weight to 7.5% by weight, based on the total weight of the adhesive composition.

Prior to curing, the adhesive composition can comprise a flowable magnetic fluid. The phrase “flowable magnetic fluid”, as used herein, generally refers to an uncured or partially cured composition which in the form of a fluid, the flow of which can be modulated (induced or restricted) via application of an external magnetic field.

Prior to curing, the adhesive composition can have a low viscosity relative to the viscosity of the adhesive composition following curing. This can allow the adhesive composition to be flowable, conformable, and readily injected or otherwise applied to a surface, for example, via a hand-powered delivery device such as a syringe.

For example, in some embodiments, the adhesive composition (prior to curing) can have a viscosity of about 1,000 cP or less (e.g., about 900 cP or less, about 800 cP or less, about 750 cP or less, about 700 cP or less, about 600 cP or less, about 500 cP or less, about 400 cP or less, about 300 cP or less, about 250 cP or less, about 200 cP or less, about 150 cP or less, about 100 cP or less, or. about 50 cP or less) at room temperature. In certain cases, the adhesive composition can have a viscosity of at least 1 cP (e.g., at least 2 cP, at least 2.5 cP, at least 5 cP, or at least 10 cP) at room temperature. The adhesive composition can have a viscosity ranging from any of the minimum values described above to any of the maximum values described above.

In some embodiments, upon curing, the adhesive composition can have a viscosity of at least 50,000 cP, at least 75,000 cP, at least 100,000 cP, at least 150,000 cP, at least 200,000 cP, or at least 250,000 cP at room temperature.

In some embodiments, the adhesive composition can be stable. Compositions are said to be stable for a given period of time when the compositions do not exhibit curing (as evidenced by an increase in viscosity of at least 25%) when stored at 25° C. in the absence of light, air, and moisture. In some embodiments, the adhesive composition can be stable at 25° C. in the absence of light, air, and moisture for at least 2 months, such as for at least 3 months, at least 6 months, at least 9 months, at least 12 months, or at least 18 months.

In some embodiments, the adhesive compositions described herein can packaged in a sealed container that excludes light, air, and moisture.

In some embodiments, upon exposure to light, air, and/or moisture (e.g., upon opening the sealed container), the adhesive composition can remain flowable under an applied magnetic field for at least 1 minute, such as for at least 5 minutes, at least 10 minutes, at least 15 minutes, or at least 30 minutes.

In some embodiments, upon exposure to light, air, and/or moisture (e.g., upon opening the sealed container), the adhesive composition can remain flowable under an applied magnetic field for from 1 minute to 2 hours, such as for from 1 minute to 1 hour, or from 1 minute to 30 minutes.

### Curable Matrix

As discussed above, the curable matrix can comprise one or more reactive precursor molecules. A variety of suitable reactive precursor molecules can be used. An appropriate combination of reactive precursor molecules can be selected depending on a number of factors, including the desired performance characteristics of the adhesive.

In some embodiments, the one or more reactive precursor molecules can comprise one or more ethylenically unsaturated monomers. Such monomers can include any ethylenically unsaturated monomer that can be polymerized by a free-radical mechanism. Suitable examples of these include, but are not limited to, (meth)acrylates, hydroxyl-containing (meth)acrylates, vinyl aromatics, vinyl halides, vinylidene halides, esters of vinyl alcohol and C₁-C₁₈ monocarboxylic acids, esters of allyl alcohol and C₁-C₁₈ monocarboxylic acids, ethylenically unsaturated monomers containing at least one carboxylic acid group, salts of ethylenically unsaturated monomers containing at least one carboxylic acid group, anhydrides of ethylenically unsaturated dicarboxylic acids, nitrites of ethylenically unsaturated carboxylic acids, ethylenically unsaturated monomers containing at least one sulfonic acid group, salts of ethylenically unsaturated monomers containing at least one sulfonic acid group, ethylenically unsaturated monomers containing at least one phosphorous atom, ethylenically unsaturated monomers containing at least one amide group, dienes, alkyds, nitrogen-containing adhesion monomers, glycidyl esters of ethylenically unsaturated monomers, vinyl esters of the formula —CH₂═CH—O—(CO)—C—(R₁₀₀)₃ wherein R₁₀₀ is an alkyl (sold under the trade name VEOVA™ by Shell), alkylaminoalkyl group-containing (meth)acrylic monomers, alkyl esters of (meth)acrylic acid containing an ether bond in the alkyl, urethane esters of (meth)acrylic acid, urea esters of (meth)acrylic acid, vinyl monomers, isocyanate esters of (meth)acrylic acid, carbonyl containing monomers, monomers containing hydrolyzable Si-organic bonds, vinyl esters of neo acids (such as those sold under the trade name EXXAR™ NEO 10 and NEO 12 from Exxon), enamines, alkyl crotonates, phosphate (meth)acrylates, and (meth)acryloxy benzophenones.

In some embodiments, the ethylenically unsaturated monomer can comprise a (meth)acrylate monomer.

In some embodiments, the ethylenically unsaturated monomer can comprise a bifunctional monomer (e.g., a bifunctional urethane monomer). In certain embodiments, the bifunctional urethane monomer can comprise a urethane di(meth)acrylate. The urethane di(meth)acrylate can be any suitable urethane polymer or oligomer which is functionalized by two (meth)acrylate moieties. In some embodiments, the urethane di(meth)acrylate can comprise a urethane chain (e.g., a urethane polymer or oligomer) appended by two terminal (meth)acrylate groups.

Urethane (meth)acrylates can undergo rapid curing/polymerization via the (meth)acrylate moieties while the urethane linkage provides adhesive properties similar to those of polyurethane-based adhesives. Further, the urethane linker promotes water sorption, helpful to initiate curing of compositions that include a water-activated initiator, and can participate in interchain hydrogen bonding to strengthen the composite structure of the cured adhesive. Urethane (meth)acrylates can be mixed with other ethylenically unsaturated monomers, such as methyl methacrylate or ethylene dimethacrylate, to optimize the viscosity and performance of the final adhesive product.

In some embodiments, the urethane di(meth)acrylate can be defined by the formula below

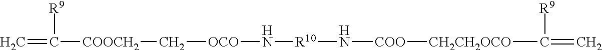

wherein
R⁹ is hydrogen or methyl; and
R¹⁰ is selected from alkylene, haloalkylene, heteroalkylene, haloheteroalkylene, cycloalkylene, alkylcycloalkylene, cycloalkylalkylene, heterocyclylene, alkylheterocyclylene, heterocyclylalkylene, arylene, alkylarylene, or alkylarylalkylene, each optionally substituted with one or more substituents individually selected from R⁸.

In certain embodiments, the urethane di(meth)acrylate is defined by the formula below

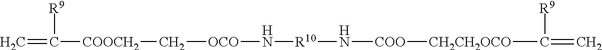

wherein
R⁹ is hydrogen or methyl; and
R¹⁰ is an alkylene group (e.g., a C₁-C₁₂ alkylene group) optionally substituted with one or more substituents individually selected from R⁸.

In certain embodiments, R¹⁰ can be the alkylene group below

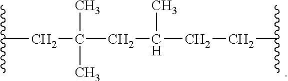 [SMILES: *CCC(C)CC(C)(C)C*]

Other suitable multi-functional (meth)acrylate components include difunctional hydrophilic (water dispersible) ethoxylated Bisphenol A di(meth)acrylates, preferably having about 10 to about 30 ethoxy groups, ethoxylated tetrabromo bisphenol A diacrylates, preferably having about 10 to about 30 ethoxy groups, polyethylene glycol di(meth)acrylates, preferably having about 200 to 600 ethylene glycol groups, metallic di(meth)acrylates, highly propoxylated glyceryl tri(meth)acrylates, preferably having about 10 to about 30 propoxy groups, trifunctional monomers of pentaerythritol tri(meth)acrylate, tetrafunctional monomers of pentaerythritol tetra(meth)acrylate, pentafunctional monomers of pentaerythritol penta(meth)acrylate, pentaerythritol dimethacrylate (PEDM), dipentaerythritol penta(meth)acrylate (DPEPA), trimethylolpropane tri(meth)acrylate (TMPTMA), ethoxylated trimethylolpropane tri(meth)acrylates, preferably having about 10 to about 30 ethoxy groups, di-trimethylolpropane tetraacrylate, tris(2-hydroxyethyl) isocyanurate, glycerol di(meth)acrylate, triethylene glycol dimethacrylate (TEGDMA), the diglycidyl (meth)acrylate adduct of Bisphenol A (Bis-GMA), or a combination thereof.

In some embodiments, the one or more reactive precursor molecules can comprise an alkyleneoxy di(meth)acrylate (e.g., a PEG di(meth)acrylate or a PPO di(meth)acrylate). In certain examples, the alkyleneoxy di(meth)acrylate can have a molecular weight of from 250 Da to 1,000 Da.

In some examples, the alkyleneoxy di(meth)acrylate can be defined by the formula below

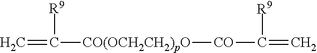

wherein
R⁹ is hydrogen or methyl; and
p is an integer from 1 to 40.

In some embodiments, the one or more reactive precursor molecules can comprise one or more monofunctional (meth)acrylates. These (meth)acrylates can be reaction products of ethylenically unsaturated carboxylic acids and C₁-C₁₈ alcohols. Examples of such (meth)acrylates include, but are not limited to, methyl (meth)acrylate, ethyl (meth)acrylate, propyl (meth)acrylate, n-butyl (meth)acrylate, isobutyl (meth)acrylate, t-butyl (meth)acrylate, pentyl (meth)acrylate, hexyl (meth)acrylate, cyclohexyl (meth)acrylate, benzyl (meth)acrylate, 2-ethylhexyl (meth)acrylate, heptyl (meth)acrylate, n-octyl (meth)acrylate, nonyl (meth)acrylate, decyl (meth)acrylate, undecyl (meth)acrylate, dodecyl (meth)acrylate, tridecyl (meth)acrylate, lauryl (meth)acrylate, stearyl (meth)acrylate, isobornyl (meth)acrylate, norbornyl (meth)acrylate, 4-tertbutylcyclohexyl (meth)acrylate, 3,3,5-trimethylcyclohexyl (meth)acrylate, dimethyl maleate, n-butyl maleate, t-butylaminoethyl (meth)acrylate, 2-t-butylaminoethyl (meth)acrylate, glyceryl (meth)acrylate, and N,N-dimethylaminoethyl (meth)acrylate.

The one or more reactive precursor molecules can comprise one or more functional ethylenically unsaturated monomers, including ethylenically unsaturated monomers containing at least one carboxylic acid group, ethylenically unsaturated monomers containing at least one hydroxy group, ethylenically unsaturated monomers containing at least one amine, ethylenically unsaturated monomers containing at least one amide, ethylenically unsaturated monomers containing at least one sulfonic acid group,

Examples of ethylenically unsaturated monomers containing at least one carboxylic acid group include, but are not limited to, (meth)acrylic acid, maleic acid, fumaric acid, itaconic acid, ethacrylic acid, crotonic acid, citraconic acid, cinnamic acid, phthalic acid, isophthalic acid, terephthalic acid, tetrahydrophthalic acid, hexahydrophthalic acid, tetrabromophthalic acid, trimellitic acid, pyromellitic acid, 1,4,5,6,7,7-hexachloro-5-norbornene-2,3-dicarboxylic acid, succinic acid, 2,6-naphthalenedicarboxylic acid, glutaric acid, sebacic acid, azelaic acid, 1,4-cyclohexanedicarboxylic acid, and 1,3-cyclohexanedicarbocylic acid.

Examples of anhydrides of ethylenically unsaturated dicarboxylic acids include, but are not limited to, maleic anhydride, succinic anhydride, phthalic anhydride, tetrahydrophthalic anhydride, hexahydrophthalic anhydride, tetrabromophthalic anhydride, trimellitic anhydride, pyromellitic anhydride, and 1,4,5,6,7,7-hexachloro-5-norbornene-2,3-dicarboxylic anhydride.

Examples of esters of ethylenically unsaturated monomers containing at least one carboxylic acid group include, but are not limited to, methylhydrogen fumarate, benzyl hydrogen maleate, butyl hydrogen maleate, octyl hydrogen itaconate, dodecyl hydrogen citraconate, butyl fumarate, octyl fumarate, octyl maleate, dibutyl maleate, and dioctyl maleate.

Examples of esters of vinyl alcohol and C₁-C₁₈ monocarboxylic acids include, but are not limited to, vinyl acetate, vinyl acetoacetate, t-butylacetoacetate, ethylacetoacetate, vinyl propionate, vinyl n-butyrate, vinyl heptanoate, vinyl perlogonate, vinyl 3,6-dioxaheptanoate, vinyl 3,6,9-trioxanundecanote, vinyl laurate, and vinyl stearate. Examples of esters of allyl alcohol and C₁-C₁₈ monocarboxylic acids include, but are not limited to, allyl acetate, allyl propionate, allyl (meth)acrylate, allyl n-butyrate, allyl laurate, allyl stearate, diallyl maleate, and diallyl fumarate.

Examples of suitable nitriles of ethylenically unsaturated carboxylic acids include, but are not limited to, acrylonitrile and methacrylonitrile. Examples of vinyl aromatics include, but are not limited to, styrene, α-methyl styrene, o-chlorostyrene, chloromethyl styrene, α-phenyl styrene, styrene sulfonic acid, salts of styrene sulfonic acid, para-acetoxystyrene, divinylbenzene, diallyl phthalate, vinyl toluene, and vinyl naphthalene. Examples of dienes include, but are not limited to, butadiene, isoprene, and chloroprene.

Examples of unsaturated monomers having both olefinic unsaturation and terminal —SO₃ groups, such as an —SO₃H group, include vinyl sulfonic acid, arylsulfonic acid, acryloyloxybenzenesulfonic acid, (meth)acryloyloxynaphthalenesulfonic acid, 2-acrylamido-2-methylpropanesulfonic acid, 2-acrylamido-2-methyl-propanesulfonic acid (AMPS), 2-sulfoethyl methacrylate (SEM) and its derivatives, 2-sulfopropyl (meth)acrylate, 4-sulfobutyl (meth)acrylate, 3-sulfobutyl (meth)acrylate, 3-bromo-2-sulfopropyl (meth)acrylate, 3-methoxy-1-sulfo-propyl (meth)acrylate, 1,1-dimethyl-2-sulfoethyl (meth)acrylamide, 3-sulfopropyl methacrylate (SPM), and active derivatives (esters and salts) of the foregoing. A combination comprising at least one of the foregoing acids, esters, or salts may also be used. Examples of derivatives include sulfonic acid salts of AMPS, SEM, and SPM, and hydrolytically active esters of AMPS, SEM, and SPM. AMPS compounds are available from Lubrizol Corporation, Wickliffe, Ohio. SEM and SPM compounds are available from Polyscience, Inc., Pa

Examples of unsaturated monomers containing at least one amide group include, but are not limited to, (meth)acrylamide, dimethyl (meth)acrylamide, N-alkyl (meth)acrylamide, N-butylacrylamide, tetramethylbutylacrylamide, N-alkylol (meth)acrylamide, N-methylol (meth)acrylamide, N-octyl acrylamide, methylene bis acrylamide, diacetoneacrylamide, ethyl imidazolidon (meth)acrylate, and N,N-dimethylaminopropylmethacrylamide.

Examples of hydroxyl containing (meth)acrylates include, but are not limited to, 2-hydroxyethyl (meth)acrylate, hydroxypropyl (meth)acrylates, hydroxypropylmethacrylates, and hydroxybutyl (meth)acrylates.

The one or more reactive precursor molecules can comprise a copolymerizable adhesion promoter. In some cases, the copolymerizable adhesion promoter can comprise an ethylenically unsaturated monomer containing at least one phosphorous atom. Examples of copolymerizable adhesion promoters include but are not limited to N-tolyglycine-N-glycerol methacrylate, pyromellitic acid dimethacrylate (PMDM), dipentaerythritol-pentaacrylate-phosphoric acid ester (PENTA), bis(2-ethylhexyl)hydrogen phosphate, 2-(methacryloyloxy)-ethyl phosphate, a butane tetracarboxylic acid-bis-hydroxyethylmethacrylate (TCB resin), methacrylic acid, maleic acid, p-vinylbenzoic acid, 11-methacryloyloxy-1,1-undecanedicarboxylic acid, 1,4-dimethacryloyloxyethylpyromellitic acid, 6-methacryloyloxyethylnaphthalene-1,2,6-tricarboxylic acid, 4-methacryloyloxymethyltrimellitic acid and the anhydride thereof, 4-methacryloyloxyethyltrimellitic acid (“4-MET”) and an anhydride thereof (“4-META”), 4-(2-hydroxy-3-methacryloyloxy)butyltrimellitic acid and an anhydride thereof, 2,3-bis(3,4-dicarboxybenzoyloxy)propyl methacrylate, methacryloyloxytyrosine, N-methacryloyloxyphenylalanine, methacryloyl-p-aminobenzoic acid, an adduct of 2-hydroxyethyl methacrylate with pyromellitic dianhydride (PMDM), an adduct of 2-hydroxyethyl methacrylate with 3,3′,4,4′-benzophenonetetracarboxylic dianhydride (BTDA) or 3,3′,4,4′-biphenyltetracarboxylic dianhydride, BPDM, which is the reaction product of an aromatic dianhydride with an excess of 2-HEMA (2-hydroxyethyl methacrylate), the reaction product of 2-HEMA with ethylene glycol bistrimellitate dianhydride (EDMT), the reaction product of 3,3′,4,4′-diphenylsulfone tetracarboxylic dianhydride and 2-HEMA (DSDM), the adduct of pyromellitic dianhydride with glycerol dimethacrylate (PMGDM), 2-(methacryloyloxy)alkyl phosphates such as 2-(methacryloyloxy)ethyl phosphate, or a combination comprising at least one of the foregoing adhesion promoters. Examples of copolymerizable nitrogen-containing adhesion promoters include, but are not limited to, ureido (meth)acrylates, (meth)acrylates with at least one of urea and thiourea in the side chains, acrylic allophanes, aminoethyl acrylate and methacrylate, dimethylaminoethyl acrylate and methacrylate, diethylaminoethyl acrylate and methacrylate, dimethylaminopropyl acrylate and methacrylate, 3-dimethylamino-2,2-dimethylpropyl acrylate and methacrylate, 2-N-morpholinoethyl acrylate and methacrylate, 2-N-piperidinoethyl acrylate and methacrylate, N-(3-dimethylaminopropyl)acrylamide and -methacrylamide, N-dimethylaminoethylacrylamide and -methacrylamide, N-diethylaminoethylacrylamide and -methacrylamide, N-(4-morpholinomethyl)acrylamide and -methacrylamide, vinylimidazole and also monoethylenically unsaturated derivatives of ethyleneurea, such as N-(2-(meth)acryloyloxyethyl)ethyleneurea, N-(β-acrylamidoethyl)ethyleneurea, N-2-(allylcarbamato)aminoethylimidazolidinone, N-vinylethyleneurea, N-(3-allyloxy-2-hydroxypropyl)aminoethylethyleneurea, N-vinyloxyethyleneurea, N-methacryloyloxyacetoxyethylethyleneurea, N-(acrylamidoethylene)ethyleneurea, N-(methacrylamidoethylene)-ethyleneurea, 1-(2-methacryloyloxyethyl)imidazolin-2-one, N-(methacrylamidoethyl)ethyleneurea, and combinations thereof.

In some embodiments, the one or more reactive precursor molecules can comprise one or more cyanoacrylate monomers. In some embodiments, the cyanoacrylate monomer(s) can be an alkyl cyanoacrylate monomer. In some embodiments, the cyanoacrylate monomer can be short alkyl chain cyanoacrylates such as methyl-, ethyl-, and isopropylcyanoacrylates. In some embodiments, the cyanoacrylate monomer(s) can be longer-chain cyanoacrylate monomer(s) such as n-butyl cyanoacrylate, ethyl cyanoacrylate, 2-octyl cyanoacrylate, or 2-octyl cyanoacrylate.

In some embodiments, the curable matrix can comprise a multicomponent composition which crosslinks to form a polymeric matrix. For example, the curable matrix can comprise a first precursor molecule and a second precursor molecule. “Precursor molecule”, as used herein, generally refers to a molecule present in the curable matrix which interacts with (e.g., crosslinks with) other precursor molecules of the same or different chemical composition in the curable matrix to form a polymeric matrix. Precursor molecules can include monomers, oligomers and polymers which can be crosslinked covalently and/or non-covalently.

In some cases, the curable matrix comprises one or more oligomeric or polymeric precursor molecules. For example, precursor molecules can include, but are not limited to, polyether derivatives, such as poly(alkylene oxide)s or derivatives thereof, polysaccharides, peptides, and polypeptides, poly(vinyl pyrrolidinone) (“PVP”), poly(amino acids), and copolymers thereof.

The precursor molecules can further comprise one or more reactive groups. Reactive groups are chemical moieties in a precursor molecule which are reactive with a moiety (such as a reactive group) present in another precursor molecule to form one or more covalent and/or non-covalent bonds. Examples of suitable reactive groups include, but are not limited to, active esters, active carbonates, aldehydes, isocyanates, isothiocyanates, epoxides, alcohols, amines, thiols, maleimides, groups containing one or more unsaturated C—C bonds (e.g., alkynes, vinyl groups, vinylsulfones, acryl groups, methacryl groups, etc.), azides, hydrazides, dithiopyridines, N-succinimidyl, and iodoacetamides. Suitable reactive groups can be incorporated in precursor molecules to provide for crosslinking of the precursor molecules.

In some embodiments, one or more of the precursor molecules comprises a poly(alkylene oxide)-based oligomer or polymer. Poly(alkylene oxide)-based oligomer and polymers are known in the art, and include polyethylene glycol (“PEG”), polypropylene oxide (“PPO”), polyethylene oxide-co-polypropylene oxide (“PEO-PPO”), co-polyethylene oxide block or random copolymers, poloxamers, meroxapols, poloxamines, and polyvinyl alcohol (“PVA”). Block copolymers or homopolymers (when A=B) may be linear (AB, ABA, ABABA or ABCBA type), star (AnB or BAnC, where B is at least n-valent, and n is an integer of from 3 to 6) or branched (multiple A's depending from one B). In certain embodiments, the poly(alkylene oxide)-based oligomer or polymer comprises PEG, a PEO-PPO block copolymer, or combinations thereof.

In some embodiments, one or more of the precursor molecules is defined by Formula I or Formula II

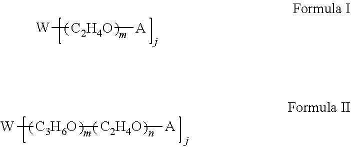

wherein
W is a branch point;
A is a reactive group (e.g., a nucleophilic group or a conjugated unsaturated group);
m and n are integers of from 1 to 500 (e.g., an integers of from 1 to 200); and
j is an integer greater than 2 (e.g., an integer of from 2 to 8).

In some embodiments, one or more of the precursor molecules comprises a biomacromolecule. The biomacromolecule can be, for example, a protein (e.g., collagen) or a polysaccharide. Examples of suitable polysaccharides include cellulose and derivatives thereof, dextran and derivatives thereof, hyaluronic acid and derivatives thereof, chitosan and derivatives thereof, alginates and derivatives thereof, and starch or derivatives thereof. Polysaccharides can derivatized by methods known in art. For example, the polysaccharide backbone can be modified to influence polysaccharide solubility, hydrophobicity/hydrophilicity, and the properties of the resultant biocompatible polymeric matrix formed from the polysaccharide (e.g., matrix degradation time). In certain embodiments, one or more of the precursor molecules comprises a biomacromolecule (e.g., a polysaccharide) which is substituted by two or more (e.g., from about 2 to about 100, from about 2 to about 25, or from about 2 to about 15) reactive groups (e.g., a nucleophilic group or a conjugated unsaturated group).

In some cases, the curable matrix can comprise a first precursor molecule which comprises an oligomer or polymer having one or more first reactive groups, each first reactive group comprising one or more pi bonds, and a second precursor molecule comprises an oligomer or polymer having one or more second reactive groups, each second reactive group comprising one or more pi bonds. The first reactive group can be reactive (e.g., via a Click chemistry reaction) with the second reactive group, so as to form a covalent bond between the first precursor molecule and the second precursor molecule. For example, the first reactive group and the second reactive group undergo a cycloaddition reaction, such as a [3+2]cycloaddition (e.g., a Huisgen-type 1,3-dipolar cycloaddition between an alkyne and an azide) or a Diels-Alder reaction.

In some cases, the curable matrix can comprise a first precursor molecule which comprises an oligomer or polymer having one or more nucleophilic groups (e.g. amino groups, thiol groups hydroxy groups, or combinations thereof), and a second precursor molecule which comprises an oligomer or polymer having one or more conjugated unsaturated groups (e.g., vinyl sulfone groups, acryl groups, or combinations thereof). In such cases, the first precursor molecule and the second precursor molecule can react via a Michael-type addition reaction. Suitable conjugated unsaturated groups are known in the art, and include those moieties described in, for example, U.S. Patent Application Publication No. US 2008/0253987 to Rehor, et al., which is incorporated herein by reference in its entirety.

In certain embodiments, the curable matrix can comprise a first precursor molecule and a second precursor molecule. The first precursor molecule comprises a poly(alkylene oxide)-based oligomer or polymer having x nucleophilic groups, wherein x is an integer greater than or equal to 2 (e.g., an integer of from 2 to 8, or an integer of from 2 to 6). The poly(alkylene oxide)-based polymer can comprise, for example, poly(ethylene glycol). The nucleophilic groups can be selected from the group consisting of sulfhydryl groups and amino groups. The first precursor molecule can have a molecular weight of from about 1 kDa to about 10 kDa (e.g., from about 1 kDa to about 5 kDa). In some embodiments, the first precursor molecule comprises pentaerythritol poly(ethylene glycol)ether tetrasulfhydryl.

The second precursor molecule can comprise a biomacromolecule having y conjugated unsaturated groups, wherein y is an integer greater than or equal to 2 (e.g., an integer of from 2 to 100, or an integer of from 2 to 25). The biomacromolecule can comprise a polysaccharide, such as dextran, hyaluronic acid, chitosan, alginate, or derivatives thereof. The conjugated unsaturated groups can be selected from the group consisting of vinyl sulfone groups and acryl groups. The second precursor molecule can have a molecular weight of from about 2 kDa to about 250 kDa (e.g., from about 5 kDa to about 50 kDa). In some embodiments, the second precursor molecule comprises dextran vinyl sulfone.

If desired, the curable matrix can further comprise one or more additional components.

In some embodiments, the crosslinking of the precursor molecules can take place under basic conditions. In these embodiments, the curable matrix can further include a base to activate the crosslinking of the precursor molecules. A variety of bases comply with the requirements of catalyzing, for example, Michael addition reactions. Suitable bases include, but are not limited to, tertiary alkyl-amines, such as tributylamine, triethylamine, ethyldiisopropylamine, or N,N-dimethylbutylamine. For a given composition (and mainly dependent on the type of precursor molecules), the curing time can be dependent on the type of base and of the pH of the solution. Thus, in these embodiments the curing time of the composition can be controlled and adjusted to the desired application by varying the pH of the basic solution.

In some embodiments, the base, as the activator of the covalent crosslinking reaction, is selected from aqueous buffer solutions which have their pH and pK value in the same range. The pK range can be between 9 and 13. Suitable buffers include, but are not limited to, sodium carbonate, sodium borate and glycine. In one embodiment, the base is sodium carbonate.

The curable matrix can further contain organic and/or inorganic additives, such as thixotropic agents, plasticizers, stabilizers, antioxidants, dyes, light stabilizers, fillers, initiators, drying agents, surface-active additives, anti-foaming agents, dye pigments, fragrances, preservatives, and combinations thereof.

In some embodiments, the curable matrix can comprise a commercially available or conventional adhesive composition. In some embodiments, the curable matrix can comprise a surgical adhesive. Examples of suitable surgical adhesive include, but are not limited to, cyanoacrylates, DURASEAL™, TRUFILL® n-BCA, BIOGLUE™ surgical adhesive, and Med A.

### Magnetic Particles

As discussed above, the magnetic particles can each comprise a magnetic particle core and shell at least partially encapsulating the magnetic particle core.

In some embodiments, the magnetic particles can have an average particle size of less than 1 micron. For example, the magnetic particles can have an average particle size of from 1 nm to 1 micron, from 10 nm to 1 micron, from 50 nm to 1 microns, from 100 nm to 1 micron, from 250 nm to 1 micron, from 500 nm to 1 micron, from 1 nm to 500 nm, from 1 nm to 250 nm, from 1 nm to 100 nm, from 1 nm to 50 nm, from 5 nm to 500 nm, from 5 nm to 250 nm, from 5 nm to 100 nm, from 5 nm to 50 nm, from 5 nm to 25 nm, from 10 nm to 500 nm, from 10 nm to 300 nm, from 10 nm to 100 nm, from 10 nm to 50 nm, or from 20 nm to 200 nm.

In certain embodiments (e.g., to produce flowable magnetic fluids), the magnetic particles can have an average particle size, as determined by electron microscopy, of from 5 nm to 50 nm (e.g., from 5 nm to 30 nm, from 5 nm to 25 nm, or from 5 nm to 20 nm.

Magnetic Particle Core. The magnetic particle core can include iron, cobalt, zinc, cadmium, nickel, gadolinium, chromium, copper, gold, silver, platinum, manganese, metal oxide, or an alloy thereof. In certain embodiments, the magnetic particle core can include iron oxide (i.e. Fe₃O₄ or Fe₂O₄). In other embodiments, the magnetic particle can include a first coating including a layer of silicon; polymer; or a metal including gold, silver, iron, cobalt, zinc, cadmium, nickel, gadolinium, chromium, copper, and manganese, or an alloy thereof. In some embodiments, the magnetic particle core is a magnetite particle. In some embodiments, the magnetic particle core can be a commercial magnetic particle, such as NanoXact Magnetite Nanoparticles® or Super Mag Silica Beads®.

Shell. The shell at least partially encapsulates (surrounds) the magnetic particle core. In certain embodiments, the shell can completely encapsulate the magnetic particle core.

The shell can be inert (and by extension render the magnetic particles inert), meaning that the magnetic particles do not induce curing of the curable matrix in the absence of light, air, and moisture. In some embodiments, the shell can improve the dispersibility of the magnetic particles in the curable matrix. For example, magnetic particles comprising the shell can provide a more stable homogenous dispersion in the curable matrix than otherwise identical magnetic particles lacking the shell. In some embodiments, the shell can be biocompatible.

In some embodiments, the shell can comprise silica, a silane, a silicone, and/or a fluoropolymers such as polytetrafluoroethylene, fluorinated ethylene propylene, perfluoroalkoxy alkane, polyvinylidene fluoride, or any combination thereof.

In some embodiments, the shell is formed by reaction of the magnetic particles with a silane defined by the following formula:

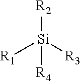

wherein R₁-R₃ are independently hydrogen, hydroxy, substituted or unsubstituted alkyl, alkenyl, cycloalkyl, alkoxy, or halogen; and
R₄ is an alkoxy or a halogen.

For example, the shell is formed by reaction of the magnetic particles with a silane such as n-octyldimethylchlorosilane, tert-butyltrichlorosilane, butyl(chloro)dimethyl silane, chlorotirmethylsilane, chlorodimethylethyl silane, chlorotributylsilane, chloro(dimethyl) isopropyl, chloro(dodecyl)dimethyl silane, methoxy(dimethyl)octylsilane, or timethoxy(octadecyl)silane.

In some embodiments, the shell can have a thickness of from 1 nm to 250 nm.

### Methods of Using

The adhesive compositions described herein can be used to adhere surfaces. The incorporated magnetic particles can be used to control application and/or curing of these adhesive compositions at a desired location. For example, these adhesive compositions can be applied as a flowable fluid, and subsequently directed to flow to a desired location prior to curing (and/or held at a desired location during curing). In some examples, the magnetic field can be applied to direct the flowable fluid to a hard-to-reach location prior to curing (e.g., a location to which the adhesive composition cannot be directly applied, for example, due to spatial constraints). In some embodiments, the magnetic field can be applied to retain the adhesive at a desired location during curing of the adhesive composition (e.g., to prevent an adhesive from being dislodged or washed away prior to curing).

Disclosed are methods for adhering a first surface and a second surface at a location that comprise applying an adhesive composition described herein as a flowable magnetic fluid to a locus in proximity to the first surface, the second surface or a combination thereof, applying a magnetic field for a period of time effective to direct the flowable magnetic fluid to the location and/or to immobilize the flowable magnetic fluid at the location; and curing the adhesive composition.

The adhesive compositions described herein can be used in many different applications, such as, but not limited to, industrial, medical, and household applications. For example, the adhesive compositions can be used in electronics, aircraft and automotive assembly, additive manufacturing, among others. For example, the adhesive compositions can be used in any application wherein a conventional cyanoacrylate adhesive is used. In some embodiments, the adhesives may be used to seal internal wounds (e.g., an artery incision), as well as external wounds (e.g., skin cuts, punctures, and lacerations).

In some embodiments the adhesive compositions described herein can be used in cardiac surgery, general wound closure, hernia surgery, artheroscopic surgery, endoscopic surgery, and any type of major or minor surgery. Further, the adhesive compositions can be used as an internal sealant, for such applications as sealing tissue together, among others.

Accordingly, also disclosed are methods of sealing a wound comprising applying an adhesive composition described herein as a flowable magnetic fluid to a locus in proximity to a tissue surface in proximity to the wound; applying a magnetic field for a period of time effective to direct the flowable magnetic fluid towards the wound and/or to immobilize the flowable magnetic fluid in place at the wound; and curing the adhesive composition to seal the wound. Optionally, these methods can further involve imaging the particles to confirm placement of the cured adhesive composition.

In some embodiments, the adhesive is applied to the desired tissue area or to an area near a hard to reach location as a flowable magnetic fluid which polymerizes/cures after a period of time of at least 25 minutes. For example, curing time last a period of time of from 25 minutes to 3 weeks, from 25 minutes to 1 hour, from 25 minutes to 4 hours, from 1 hour to 8 hours, from 25 minutes to 12 hours, from 25 minutes to 24 hours, from 25 minutes to 36 hours, from 25 minutes to 48 hours, from 25 minutes to 3 days, from 25 minutes to 1 week, from 25 minutes to 2 weeks. In some embodiments, the curing time last a period of time of about 25 minutes. In some embodiments, curing time last a period of time of about 48 hours. In some embodiments, curing time last a period of time of about 3 weeks.

The polymerized patch of adhesive allows the tissue to heal properly. The components of the adhesive then are cleared from the body.

In some embodiments, the magnetic field is applied for a period of time of at least 25 minutes. For example, the magnetic field is applied for a period of time of from 25 minutes to 3 weeks, from 25 minutes to 1 hour, from 25 minutes to 4 hours, from 1 hour to 8 hours, from 25 minutes to 12 hours, from 25 minutes to 24 hours, from 25 minutes to 36 hours, from 25 minutes to 48 hours, from 25 minutes to 3 days, from 25 minutes to 1 week, from 25 minutes to 2 weeks. In some embodiments, the magnetic field is applied for about 25 minutes. In some embodiments, the magnetic field is applied for about 48 hours. In some embodiments, the magnetic field is applied for about 3 weeks.

By way of non-limiting illustration, examples of certain embodiments of the present disclosure are given below.

### EXAMPLES

A series of experiments were conducted to develop magnetic superglues. The magnetic superglues evaluated include a suspension of magnetic particles dispersed within in a curable adhesive matrix. The compositions can exhibit prolonged curing times after the addition of magnetic particles (i.e., they can function as stable magnetic superglues).

Various magnetic particles were tested, including ‘oil soluble’ and ‘water soluble’ particles. Here, ‘soluble’ refers to suspension stability of the magnetic particles in the indicated fluid, oil or water. Dry magnetic particles were also tested. Magnetic particles failed to achieve a suspension or failed to maintain superglue in a liquid (uncured) state. Control studies were conducted to demonstrate that the curing did not result from exposure to the atmosphere (e.g., humid air) during the addition of magnetic particles.

Several experiments were conducted to determine if magnetic particles could be developed that (1) achieved colloidal stability or re-suspension with agitation; and (2) maintained the superglue in a fluid (uncured) state. Magnetic particles of different materials, different sizes, and with different surface properties were tested. Molecular substitutions at the surface of magnetic particles were also tried. Silica-coated magnetic particles were able to achieve colloidal stability or resuspension with agitation and maintained superglue in a fluid state (uncured). Such compositions of stable magnetic superglues would be ‘ready to use’ and would not require mixing of magnetic particles and superglue, for example.

### Materials and Methods

Magnetic Particles. Four commercially available magnetic particles were used for initial investigation: EFH Ferrofluid (oil suspension), commercially available from Ferrotec Corporation; EMG 700 Ferrofluid (water suspension), commercially available from Ferrotec Corporation; NanoXact Magnetite nanoparticles (PVP coated; 20 nm), commercially available from nanoComposix; and Super Mag Silica Beads (200 nm; water suspension), commercially available from Ocean NanoTech.

Cyanoacrylate glues. Four commercially available cyanoacrylate adhesives were used for initial investigation: VETBOND, an n-butyl cyanoacrylate adhesive commercially available from 3M; APOLLO 2611, an ethyl cyanoacrylate adhesive commercially available from H.B. Fuller/Cyberbond; DERMABOND ADVANCED, a 2-octyl cyanoacrylate adhesive commercially available from Johnson & Johnson; and CHENSO 1640, a 2-octyl cyanoacrylate adhesive commercially available from Chenso.

### Preparation of Magnetic Cyanoacrylate Adhesives

Preparation of magnetic cyanoacrylate glues with EFH Ferrofluid. EFH Ferrofluid (10 μL) was added to 90 μL of each of the cyanoacrylate glues above. The resulting solutions were then mixed by vortexing for 30 seconds. The resulting mixtures were stored at room temperature. All adhesives cured in less than 5 minutes.

Preparation of magnetic cyanoacrylate glues with purified EFH Ferrofluid particles. EFH Ferrofluid was purified to remove surfactants, such as oleic acid. Briefly, 5 ml of EFH Ferrofluid was suspended in 45 mL acetone by vortexing for 30 seconds. The magnetic particles precipitated from acetone solution. The precipitates were collected using a rare earth magnet, and the supernatant was removed. The isolated particles were first suspended in 5 ml dichloromethane and then added to 45 mL acetone with vortexing. The magnetic particles precipitated from acetone, and were again collected using a rare earth magnets. This purification process was repeated 3 times. The collected magnetic particles were dried in a vacuum oven overnight. 10 mg of the dried magnetic particle powder was then suspended in 90 μL of the cyanoacrylate glues above by vortexing for 30 seconds. The resulting mixtures were then stored at room temperature. All adhesives cured in less than 30 minutes.

Preparation of magnetic cyanoacrylate glues with NanoXact Magnetite Nanoparticles. 10 mg of NanoXact Magnetite Nanoparticles were suspended in 90 μL of the cyanoacrylate glues above by vortexing for 30 seconds. The resulting mixtures were then stored at room temperature. All adhesives cured in less than 30 minutes.

Surface modification of Super Mag Silica Beads (200 nm) with octyl groups and preparation of magnetic cyanoacrylate glues with these surface modified beads. Super Mag Silica Beads (10 mg/mL) were collected from 10 mL of solution using a rare earth magnet. The supernatant was removed. The collected particles were re-suspended in 10 mL H₂O, and then collected using a rare earth magnet. This washing process was repeated 2 more times. The collected magnetic beads were lyophilized. The lyophilized powder was suspended in 6 mL dry dichloromethane by stirring. Then 60 μL 1-methyl-2-pyrrolidinone and 100 μL n-octyldimethylchlorosilane was added. The mixture was then reacted at 50° C. for 24 hours. The resulting octyl-modified magnetic beads were collected using a magnet and washed by dichloromethane three times. 10 mg of octyl-modified Super Mag Silica Beads were then added to 90 μL of each of the cyanoacrylate glues above. The resulting solutions were then mixed by vortexing for 30 seconds. The resulting mixtures were stored at room temperature. All adhesives cured after 48 hours.

Surface modification of NanoXact Magnetite Nanoparticles (20 nm) with silica shells and octyl groups and preparation of magnetic cyanoacrylate glues with these surface modified particles. 100 mg of NanoXact Magnetite Nanoparticles were dissolved in 15 mL ethanol. 0.3 mL NH₄OH solution (30%) and 90 μL of TEOS were added then added to the solution. The mixture was then reacted at room temperature for 24 hours. The resulting particles were collected using a magnet, and dried under vacuum. The resulting dried powder was then suspended in 5 mL DMF, and 10 mL dichloromethane and 90 μL of n-octyldimethylchlorosilane were added. The mixture was then reacted at 50° C. for 24 hours. The particles were then washed with dichloromethane 3 times, and dried under vacuum. 10 mg of the octyl-modified silica coated NanoXact Magnetite Nanoparticles was then added to 90 μL of each of the cyanoacrylate glues above. The resulting solutions were then mixed by vortexing for 30 seconds. The resulting mixtures were stored at room temperature. All adhesives started to cure after 3 weeks.

Table 1 shows the results of adding the octyl magnetic particle (octyl-MNP) suspension in various superglues.

| Glues | Description | Suspension | Curing time | Magnetic test |
|---|---|---|---|---|
| 3M Vetbond | n-Butyl | Octyl-MNPs | No curing | Suspension |
| | cyanoacrylate | content <5 wt %; | observed | could not be |
| | Low viscosity | Powder partially | for 48 h | attracted by |
| | | suspended; | | magnetic field |
| Dermabond | 2-Octyl | Octyl-MNPs | No curing | Suspension |
| Advanced | cyanoacrylate | content <4 wt %; | observed | could be well |
| | High viscosity | Powder partially | for 48 h | attracted by |
| | | suspended; | | magnetic field; |
| CHENSO | 2-Octyl | Octyl-MNPs | No curing | Suspension |
| | cyanoacrylate | content <5 wt %; | observed | could be |
| | Low viscosity | Powder partially | for 48 h | attracted by |
| | | suspended; | | magnetic field; |
| Cyberbond 2611 | Ethyl | Octyl-MNPs | About 30 min | |
| | cyanoacrylate | content <5 wt %; | | |
| | Medium | Powder partially | | |
| | viscosity | suspended | | |

Table 2 shows the results of stable magnetic superglues with purified oil particles (EFH1), polymer encapsulated particles, and nanorods.

| | | | Curing |
|---|---|---|---|
| Particles | Glues | Suspension | time |
| Purified oil NPs | 3M Vetbond | Powder partially | about 15 min |
| (EFH1) | | suspended in glue | |
| | | (finally NPs | |
| | | concentration <3%); | |
| | | The suspended NPs | |
| | | maintain stale in | |
| | | glue under | |
| | | magnetic field. | |
| Purified oil NPs | Cyberbond | Powder barely | Less than |
| (EFH1) | 2611 | suspended in glue | 5 min |
| | | (finally NPs | |
| | | concentration <1%); | |
| | | The suspended NPs | |
| | | maintain stable in | |
| | | glue under | |
| | | magnetic field. | |
| Purified oil NPs | Dermabond | Powder barely | About 25 min |
| (EFH1) | Advanced | suspended in glue | |
| | | (finally NPs | |
| | | concentration <1%); | |
| | | The suspended NPs | |
| | | maintain stable in | |
| | | glue under | |
| | | magnetic field. | |
| Polymer | 3M Vetbond | Powder barely | Less than |
| encapsulated NPs | | suspended in glue | 10 min |
| | | (finally NPs | |
| | | concentration <1%); | |
| | | The suspended NPs | |
| | | maintain stable in | |
| | | glue under | |
| | | magnetic field. | |
| Polymer | Cyberbond | Powder barely | Less than |
| encapsulated NPs | 2611 | suspended in glue | 10 min |
| | | (finally NPs | |
| | | concentration <1%); | |
| | | The suspended NPs | |
| | | maintain stable in | |
| | | glue under | |
| | | magnetic field. | |
| Polymer | Dermabond | Powder barely | About 25 min |
| encapsulated NPs | Advanced | suspended in glue | |
| | | (finally NPs | |
| | | concentration <1%); | |
| | | The suspended NPs | |
| | | maintain stable in | |
| | | glue under | |
| | | magnetic field. | |
| Nanorods | 3M Vetbond | Powder could be | about 15 min |
| | | suspended in glue; | |
| | | the suspension is | |
| | | not stable; phase | |
| | | separation under | |
| | | magnetic field. | |
| Nanorods | Cyberbond | Powder could be | about 10 min |
| | 2611 | suspended in glue; | |
| | | the suspension is | |
| | | not stable; phase | |
| | | separation under | |
| | | magnetic field. | |

Purified oil NPs (EFH1), Polymer encapsulated NPs, and nanorods could not be well suspended in Poppyseed oil.

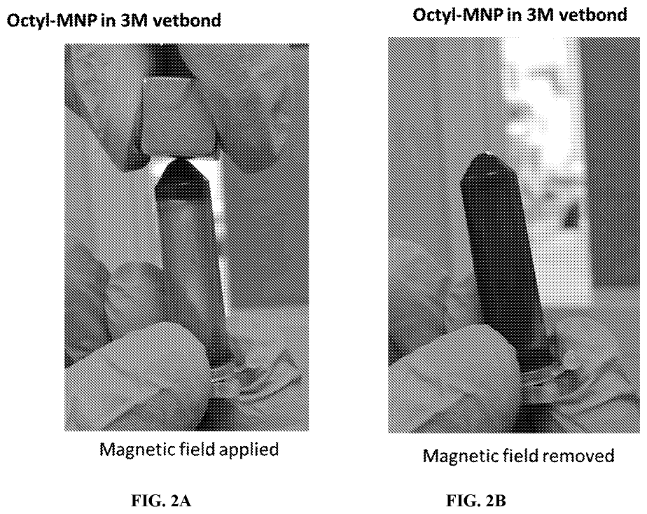

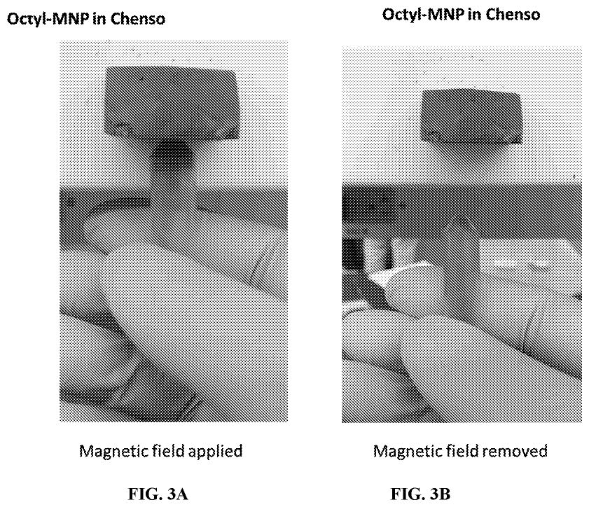

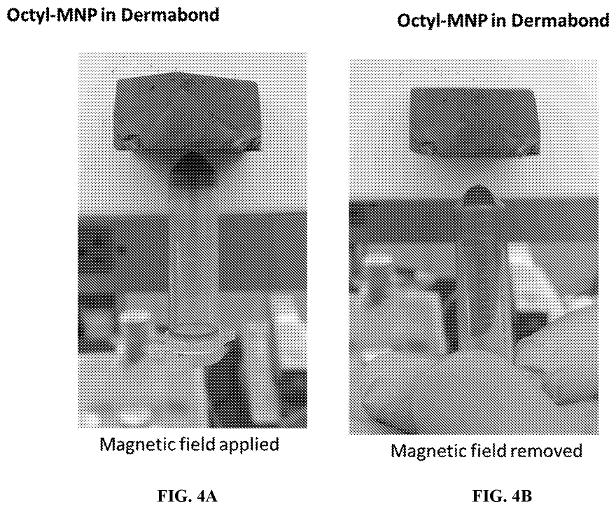

FIGS. 2A-4B show images of octyl-MNP in 3M vetbond, Chenso, and Dermabond when the magnetic field is applied and removed. While the magnetic field was applied the magnetic glue was held by magnetic field offsetting the effect of gravity. The magnetic glue flowed under the effect of gravity without the magnetic field.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. An adhesive composition comprising a curable matrix comprising one or more reactive precursor molecules, and a population of magnetic particles dispersed within the curable matrix;
wherein the magnetic particles each comprise a magnetic particle core and shell at least partially encapsulating the magnetic particle core.

2. The composition of claim 1, wherein the magnetic particles are present in an effective amount to induce and direct flow of the adhesive composition under an applied magnetic field.

3. The composition of any of claims 1-2, wherein the magnetic particles are present in an amount of from 0.1% by weight to 30% by weight, such as from 2.5% by weight to 7.5% by weight, based on the total weight of the adhesive composition.

4. The composition of any of claims 1-3, wherein the adhesive composition is stable at 25° C. in the absence of light, air, and moisture for at least 2 months, such as for at least 3 months, at least 6 months, at least 9 months, at least 12 months, or at least 18 months.

5. The composition of any of claims 1-4, wherein the adhesive composition has a cure time of at least 1 minute, such as a cure time of at least 5 minutes, a cure time of at least 10 minutes, a cure time of at least 15 minutes, or a cure time of at least 30 minutes.

6. The composition of any of claims 1-5, wherein the adhesive composition has a cure time of from 1 minute to 2 hours, such as from 1 minute to 1 hour, or from 1 minute to 30 minutes.

7. The composition of any of claims 1-6, wherein the adhesive composition is packaged in a sealed container that excludes light, air, and moisture; and
wherein upon opening the sealed container and exposing the adhesive composition to light, air, moisture, or any combination thereof, the composition remains flowable under an applied magnetic field for at least 1 minute, such as for at least 5 minutes, at least 10 minutes, at least 15 minutes, or at least 30 minutes.

8. The composition of any of claims 1-7, wherein the adhesive composition is packaged in a sealed container that excludes light, air, and moisture; and
wherein upon opening the sealed container and exposing the adhesive composition to light, air, moisture, or any combination thereof, the composition remains flowable under an applied magnetic field for from 1 minute to 2 hours, such as for from 1 minute to 1 hour, or from 1 minute to 30 minutes.

9. The adhesive of any of claims 1-8, wherein the one or more reactive precursor molecules comprise one or more ethylenically unsaturated monomers.

10. The adhesive of any of claims 1-9, wherein the one or more reactive precursor molecules comprise one or more (meth)acrylate monomers.

11. The adhesive of any of claims 1-10, wherein the one or more reactive precursor molecules comprise one or more cyanoacrylate monomers.

12. The adhesive of claim 11, wherein the one or more cyanoacrylate monomers comprise n-butyl cyanoacrylate, ethyl cyanoacrylate, 2-octyl cyanoacrylate, 2-octyl cyanoacrylate, or any combination thereof.

13. The adhesive of any of claims 1-8, wherein the one or more reactive precursor molecules comprise a first precursor molecule and a second precursor molecule.

14. The adhesive of claim 13, wherein the first precursor molecule comprise a bis-epoxide and the second precursor molecule comprises a hardener, such as an amine hardener, an acid hardener, an anhydride hardener, an alcohol hardener (e.g., a phenol hardener), a thiol hardener, or any combination thereof.

15. The adhesive of claim 13, wherein the first precursor molecule comprises an oligomer or polymer having one or more first reactive groups, each first reactive group comprising one or more pi bonds, and the second precursor molecule comprises an oligomer or polymer having one or more second reactive groups, each second reactive group comprising one or more pi bonds, and
wherein the first reactive group is reactive with the second reactive group to form a covalent bond between the first precursor molecule and the second precursor molecule.

16. The adhesive of claim 15, wherein the first reactive group and the second reactive group undergo a cycloaddition reaction.

17. The adhesive of claim 13, wherein the first precursor molecule comprises an oligomer or polymer having one or more nucleophilic groups, and the second precursor molecule comprises an oligomer or polymer having one or more conjugated unsaturated groups.

18. The adhesive of claim 17, wherein the first precursor molecule comprises a poly(alkylene oxide)-based oligomer or polymer having x nucleophilic groups, wherein x is an integer greater than or equal to 2, such as an integer of from 2 to 8 or from 2 to 6.

19. The adhesive of claim 18, wherein the poly(alkylene oxide)-based oligomer or polymer comprises poly(ethylene glycol).

20. The adhesive of any of claims 17-19, wherein the nucleophilic groups are selected from the group consisting of sulfhydryl groups and amino groups.

21. The adhesive of any of claims 15-20, wherein the first precursor molecule has a molecular weight of from about 1 kDa to about 10 kDa.

22. The adhesive of any of claims 17-21, wherein the first precursor molecule comprises pentaerythritol poly(ethylene glycol)ether tetrasulfhydryl.

23. The adhesive any of claims 17-22, wherein the second precursor molecule comprises a biomacromolecule having y conjugated unsaturated groups, wherein y is an integer greater than or equal to 2, such as an integer of from 2 to 100 or from 2 to 25.

24. The adhesive of claim 23, wherein the biomacromolecule comprises a polysaccharide, such as dextran or a derivative thereof.

25. The adhesive of any of claims 17-24, wherein the conjugated unsaturated groups are selected from the group consisting of vinyl sulfone groups and acryl groups.

26. The adhesive of any of claims 15-25, wherein the second precursor molecule has a molecular weight of from about 2 kDa to about 250 kDa, such as from about 5 kDa to about 50 kDa.

27. The adhesive of any of claims 17-26, wherein the second precursor molecule comprises dextran vinyl sulfone.

28. The adhesive of any of claims 1-27, wherein the magnetic particle core comprises iron, cobalt, zinc, cadmium, nickel, gadolinium, chromium, copper, gold, silver, platinum, manganese, metal oxide, or an alloy thereof.

29. The adhesive of any of claims 1-28, wherein the magnetic particle core comprises iron oxide.

30. The adhesive of any of claims 1-29, wherein the magnetic particle core comprises a magnetite particle.

31. The adhesive of any of claims 1-30, wherein the shell is biocompatible.

32. The adhesive of any of claims 1-31, wherein the shell is effective to render the magnetic particles inert.

33. The adhesive of any of claims 1-32, wherein the shell comprises silica, a silane, a silicone, a fluoropolymer (e.g., polytetrafluoroethane), or any combination thereof.

34. The adhesive of claim 33, wherein the shell is formed by reaction of the magnetic particles with a silane defined by the following formula: 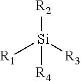
wherein R₁-R₃ are independently hydrogen, hydroxy, substituted or unsubstituted alkyl, alkenyl, cycloalkyl, alkoxy, or halogen; and
R₄ is an alkoxy or a halogen.

35. The adhesive of claim 34, wherein the shell is formed by reaction of the magnetic particles with n-octyldimethylchlorosilane.

36. The adhesive of any of claims 1-35, wherein the shell has a thickness of from 1 nm to 250 nm.

37. The adhesive of any of claims 1-36, wherein the magnetic particles have an average particle size of less than 500 nm as determined by SEM, such as an average particle size of from 50 nm to 500 nm.

38. A method for adhering a first surface and a second surface at a location, the method comprising:
applying the adhesive composition of any of claims 1-37 as a flowable magnetic fluid to a locus in proximity to the first surface, the second surface or a combination thereof,
applying a magnetic field for a period of time effective to direct the flowable magnetic fluid to the location and/or to immobilize the flowable magnetic fluid at the location; and
curing the adhesive composition.

39. A method of sealing a wound, the method comprising:
applying the adhesive composition of any of claims 1-37 as a flowable magnetic fluid to a locus in proximity to a tissue surface in proximity to the wound;
applying a magnetic field for a period of time effective to direct the flowable magnetic fluid towards the wound and/or to immobilize the flowable magnetic fluid in place at the wound;
and curing the adhesive composition to seal the wound.

40. The method of claim 39, wherein the wound is an internal wound.

41. The method of any of claims 38-40, wherein the method further comprises imaging the particles to confirm placement of the cured adhesive composition.
